(19) United States
(12) Patent Application Publication
(10) Pub. No.: US 2014/0275817 A1
(43) Pub. Date: Sep. 18, 2014

(54) Portable, Pediatric Medical Diagnostic Device

(71) Applicant: GUARDIT TECHNOLOGIES, LLC, Fairfax Station, VA (US)

(72) Inventor: Michael H. Script, Fairfax Station, VA (US)

(73) Assignee: GUARDIT TECHNOLOGIES, LLC, Fairfax Station, VA (US)

(21) Appl. No.: 13/800,389

(22) Filed: Mar. 13, 2013

(51) Int. Cl. A61B 5/113; A61B 5/00; A61B 1/227; A61B 5/1455; A61B 7/02; A61B 5/0402; A61B 5/01; A61B 5/0205

(52) U.S. Cl. CPC A61B 5/113; A61B 5/01; A61B 5/6831; A61B 5/6823; A61B 5/6824; A61B 5/02055; A61B 5/14551; A61B 7/02; A61B 5/0402; A61B 1/227; A61B 5/0006; A61B 5/0008; A61B 5/0013
USPC 600/301; 600/534; 600/484; 600/324

(57) ABSTRACT

A hand-held pediatric medical diagnostic device includes a sensor module adapted to detect one or more corresponding conditions of a child. A gel material at least partially coats a contact surface on the diagnostic device. The area of the contact surface and the weight associated therewith are selected to substantially stabilize the sensor module during use. In one version, the pediatric device comprises a pediatric respiratory rate sensing device in which an accelerometer is connected to a portable, rechargeable power source.

### FIELD

This disclosure relates to pediatric medical diagnostic devices and, in particular, to portables devices suitable for field use.

### BACKGROUND

The diagnosis of pediatric medical conditions in remote areas, in developing countries, in war-torn areas, or in other such “field” locations may present challenges to medical practitioners, relief- or aid-workers, or similar personnel. For example, typical hospital supplies may be unavailable or in short supply. Environmental factors may render traditional hospital devices inaccurate or nonfunctional, and sanitary conditions may be compromised.

In developing countries outside of hospital settings, or in other field situations, medical or relief personnel are often required to resort to counting, watches, or other basic techniques to take pediatric respiratory rates or other vital signs. Such basic or manual counting or timing methods are often not accurate or are often unable to be recorded or otherwise subsequently analyzed. Disposable medical supplies, including those related to diagnostic devices, may likewise be unavailable in remote or field applications, or in developing countries.

The age of children associated with pediatric diagnoses may cause them to squirm, cry or otherwise cause further challenges to obtaining accurate or useful readings by basic or manual counting and timing methods. All of the foregoing hampers effective treatment of medical conditions suffered by children in developing countries or in other less-than-optimal environments.

It would be desirable to address the foregoing drawbacks and disadvantages.

### SUMMARY

According to one implementation, a hand-held pediatric medical diagnostic device makes use of a sensor module to detect one or more corresponding medical conditions of a child. The sensor module has a contact surface which can be placed in operative contact with the child. The device includes a user interface to select a diagnostic program associated with the sensor module. The user interface includes a readout screen corresponding to the diagnostic procedure being performed.

In another implementation, the device comprises a respiratory sensing device with an accelerometer adapted to detect respiration of a child. A contact surface is operatively connected to the accelerometer and adapted to be placed in contact with the child's chest. A gel material at least partially coats the contact surface. One suitable gel material is silicone, but other gel materials, such as polyvinyl chloride and latex, are likewise suitable. The characteristics of the gel material and the area and weight associated with the contact surface act to substantially stabilize the accelerometer during use so as to reduce spurious signals.

### BRIEF DESCRIPTION OF THE DRAWINGS

The invention will be explained in greater detail hereinafter on the basis of exemplary implementations, and with reference to the drawing, in which:

FIG. 1 shows a front elevational view of one implementation of the present disclosure;

FIG. 2 shows a side elevational view of the implementation of FIG. 1;

FIG. 3 shows a rear elevational view of the implementation of FIGS. 1 and 2;

FIG. 4 shows another implementation of the present disclosure in partially schematic form; and

FIG. 5 shows yet another implementation of the present disclosure.

### DETAILED DESCRIPTION

Referring to the drawing, and in particular to FIGS. 1-3, a portable pediatric medical diagnostic device has been implemented as a pediatric respiratory rate sensing device 21. Sensing device 21 makes use of an accelerometer 23, such as that disclosed in U.S. Pat. No. 7,554,445, the teachings of which are incorporated herein. In general terms, device 21 is used to detect the respiration rate of a child by placing a contact surface 25 against the chest of a child, such as on the sternum. The contact surface 25 is operatively connected to accelerometer 23, meaning that accelerometer 23 detects the relative raising and lowering of contact surface 25 from inhalation and exhalation of the child's chest over a period of time, thereby determining the respiration rate.

A gel material 27, such as silicone in this implementation, is disposed at least partially covering contact surface 25. Gel material 27 assists in holding device 21 in a desired position on the child and may also reduce shocks, jolts, or other movement of device 21 and its associated accelerometer 23 from causes other than pediatric respiration or other characteristics to be measured. Gel material 27 may likewise consist of polyvinyl chloride or latex. The area and weight associated with contact surface 25, as well as the characteristics of gel material 27, are selected to substantially stabilize accelerometer 23 during use and thereby reduce spurious signals.

For example, suitable implementations may have contact surface 25 with an area ranging from about 4 square inches to about 14 square inches, and a weight of about 130 grams to about 200 grams. In the illustrated implementation, gel material 27 has an area substantially corresponding to contact surface 25, a thickness of about ⅛ inch to ¼ inch, and a tackiness sufficient, under standard atmospheric conditions, to both adhere to the skin of the child in a variety of positions and be removable therefrom without substantially harming the skin of the child.

Operation of device 21 is accomplished through a suitable control system 29 housed in a user interface module 31. Control system 21 includes a suitable user interface and a processor 28 suitably programmed to process input from the user interface and from accelerometer 23. In this implementation, user interface module 31 and its associated control system 29 include a user interface having user input areas 33 in the form of user-activatable buttons 35 corresponding to respective modes of operation of device 21. User input areas 33 may also include a suitable keypad 30 (FIG. 4). In this implementation, control system 29 is configured to sense respiratory rates in children from newborn infants to age five, and buttons 35 allow operation of the device in the following three modes: infant, toddler, and child modes. It will be appreciated that other implementations of device 21 may be configured for other pediatric or child age ranges, including children age five and above. The provision of three, easily accessible buttons 35 corresponding to three respiratory programs, lends device 21 operational simplicity, which may be advantageous under adverse field conditions or environments. User interface module 31 further includes a screen 37 for displaying readouts associated with operation of device 21. Read-outs may assume any number of forms, such as numeric, graphical, color-coding, audio or other visual indicators.

It would likewise be appreciated that interface module 31 may assume any number of alternative configurations, including having screen 37 comprise a touch screen with user-activatable areas, readouts, or any number of input and output functions, either in addition to or instead of depressible buttons 35.

Device 21 and its various components are powered by a portable, rechargeable power source 39 electrically connected to control system 29 and accelerometer 23. Power source 39 can be a replaceable battery, a rechargeable battery, a graphene capacitor, or a manually operable crank charger 41, as shown in this implementation.

Device 21 makes use of a suitable housing 43 inside of which one or more of the above-described components are carried or contained. In one implementation, contact surface 25 may comprise a lower surface of housing 43, and user interface module 31 may have its screen 37 viewable through an upper surface of housing 43. Power source 39 can be selectively connectable to housing 43, or removable therefrom, so that power source 39 can be replaced with an alternative power source, as needed. Physical attachment of power source 39 to housing 43 may also result in electrical connection of power source 39 to control system 29 and accelerometer 23 for suitable operation, such as through a suitable connector 32 (FIG. 4).

Accelerometer 23, in this implementation, is part of a sensor module 24 which optionally includes a temperature sensor 45. In order for sensor 45 to be operatively associated with the child, a suitable aperture 46 is formed in gel material 27, or other suitable conductive path may be provided. Sensor module 24 may be removably attached to user interface module 31. Power source 39, as discussed previously, may likewise assume the form of a user-swappable power module 47. In this implementation, then, sensor module 24, including accelerometer 23 and optional temperature sensor 45, and power module 47, comprising crank charger 41, are each user-detachable from module 31, to enable attachment of a second power module or a second sensor module, each potentially being different from the first such modules. As such, sensor module 24 with accelerometer 33 and option temperature sensor 45 can be substituted for another sensor module having one or more different diagnostic sensors, and, likewise, crank charger 41 can likewise be replaced with a battery, capacitor, or other suitable power source.

As best seen in FIG. 2, interface module 31 may be configured so as to connect to power module 47 at a first location and sensor module 24 at a second location on module 31. In this embodiment, respective surfaces of modules 24, 31, and 47 form contact surface 25, to which a layer of gel material 27 is applied having a thickness of about ⅛ inch to ¼ inch. The configurations of contact surface 25 and gel material 27 may be varied depending on the application or device form-factors desired. The weight of device 21, as transmitted to contact surface 25, as well as the thickness or tackiness of gel material 27, are “tuned” or otherwise selected so that sensor module 24 is in operative contact with the child being diagnosed, remains stable for a clinically sufficient period of time so that diagnosis is accomplished reasonably accurately, and is isolated so as to reduce the occurrence of spurious or inaccurate readings during such diagnosis. Gel material 27 in this implementation comprises a layer of silicone configured to be applied to contact surface 25 and selectively peeled away therefrom. The silicone layer is chosen so as to remain substantially intact, washable, and replaceable back on contact surface 25 for reuse. Other implementations may include a cap for either covering gel material 27, or an applicator for adhering a layer of gel material 27 to all or the desired portion of contact surface 25.

In some implementations, a settling in period before commencing sensing or other diagnostics may be desirable, such as a period of time from when the device is placed on the child's chest or other body part to commencement of sensor detection. Control system 29 can likewise be programmed to account for such settling time or otherwise adjust sampling periods or filter sensed input to further assure accurate readings

The operation of portable, pediatric respiratory rate sensing device 21 may be readily appreciated from the foregoing description. A medical practitioner, aid worker, or other field personnel may use the device in connection with a child to be examined. After turning on the device through power switch or other suitable means (not shown), a suitable respiratory program is selected, in this case infant, toddler, or child, by depressing corresponding user-selectable buttons 35. Contact surface 25, including gel material 27 disposed thereon, is placed on the child's chest at some point before or after the desired program is selected. The tackiness of gel material 27 permits adherence even if the child is not perfectly still, as may sometimes occur when dealing with children. The resiliently compressible characteristics of gel material 27 may likewise serve to insulate or cushion sensor module 24 from outside shocks or other unintended input which could result in spurious readings.

Readouts from the diagnostic being performed are suitably displayed on screen 37. Depending on the particular application, such readouts can be numeric, graphic, include sounds, lights, or other suitable indicators, in any suitable combination to indicate the respiratory rate.

In this implementation, simultaneous with detection of respiration by means of accelerometer 23, or as an alternative thereto, temperature sensor 45 may detect the child's temperature through suitable operative contact with the child, and a corresponding reading can be displayed in screen 37, whether numeric, color-coded, or audio-signal in nature.

If the layer of silicone on device 21 becomes dirty or otherwise loses sufficient tackiness to operatively contact the child being diagnosed, the user may peel the used gel material away from contact surface 25 and either replace it with another silicone film, or wash the soiled silicone film and return it to the contact surface.

Portable, pediatric respiratory rate sensing device 21 is just one possible implementation of a pediatric medical diagnostic device 121 shown schematically in FIG. 4, in which like reference numerals correspond to like components.

As in the case of device 21, pediatric medical diagnostic device 121 includes a sensor module 124 which may be adapted to detect not only respiratory rate or temperature as discussed with reference to the previous implementation, but may include one or more of the following sensors: a galvanic sensor, a heart rate sensor, an oximeter sensor, a stethoscope, a flow meter, an ECG/EKG sensor, an otoscope, or a photographic camera. The foregoing sensors may be housed separately in respective sensor modules, or may be combined together into one or more sensor modules, as appropriate. It will likewise be appreciated that, depending on the sensor, operative contact with different body locations of the child is contemplated. In this way, a plurality of the sensor modules 124, including those specified above, may be removably connected to a suitably multiplexed version of sensor connector 32, so as to electrically connect to control system 129 of device 121. Sensor connector 32, in this implementation, is thus suitably adapted to removably receive any selected one of sensor modules 124, so that device 121 can be used to diagnose any number of conditions of a child to be examined.

Sensor modules 124 and control system 129 include suitable programming, interfaces, connectors, or drivers to appropriately receive inputs from the child being examined, or from the selected one or more sensor modules 124, and process such inputs. Programming also allows the user to select corresponding programs through user interface module 131, and display suitable readouts through screen 137.

Medical diagnostic device 121, in this implementation, is hand-held, and includes a contact surface coated with gel material, as well as a portable, rechargeable power source electrically connected to user interface module 131, as discussed with reference to device 21. As in device 21, medical diagnostic device 121 is configured so that its area and weight associated with its contact surface, as well as the characteristics of its gel material, lead to substantial stabilization of the sensor module 121 during use.

Devices 21, 121 may include a transmitter, such as that shown at 141 in FIG. 4. Transmitter 141 is operatively connected to control system 29, 129 through a suitable communication interface 143. In either application, transmitter 141 sends signals corresponding to the condition detected by accelerometer 33 or the corresponding sensor module 24, 124 in a suitable transmission format, using a suitable antenna 148 as needed. Devices 21, 121 may make use of Bluetooth, Wi-Fi, NFC, radio, cellular, AM/FM, 802.5.14, or other suitable protocols for wireless diagnostic devices.

Devices 21, 121 may be equipped with a suitable LED 50, 150 to illuminate surroundings. Devices 21, 121 are suitably equipped not only with processing capabilities, drivers, and other software programming to operate sensor modules as discussed above, but likewise include suitable internal memory, such as flash/EPROM and SDRAM, as well as removable memory in the form of MMC/SD cards. As such, diagnostics procedures performed on one or more children may not only be transmitted by the means discussed previously, but may be stored in suitable memory for uploading or removed for use by other systems.

In a similar vein, devices 21, 121 are equipped with a suitable connection interface, such as a USB port 151, which may be used not only to charge power module 47, 147 therein, but to upload or transfer data to a computer device or computer network.

Although device 21 has been shown to include a substantially rectangular contact surface 25, it will be appreciated that any number of variations to the form of contact surface 25, as well as the overall form of device 21, are contemplated within the scope of the present disclosure. Thus, for example, in one alternative implementation, in portable medical diagnostic device 21, gel material 227 may be suitably applied to lower surfaces of a pair of resiliently flexible straps 245, as shown in FIG. 5. Straps 245 extend from housing 243, which may carry one or more of the sensors discussed previously, a suitable portable power source, and the associated user interface discussed with reference to devices 21, 121. The straps 245 may be extended so as to be in operative contact with the appropriate location on the child's body being diagnosed, such as the chest in the case of an accelerometer. The form factor of device 221 shown in FIG. 5 may be such as to be worn around either the user's wrist of the patient's wrist, with straps 245 being flexible enough wrap around and be retained on such wrist.

The described and illustrated arrangements are intended to provide a general understanding of the structure of various embodiments, and they are not intended to serve as a complete description of all the elements and features of the devices and related methods herein. Many other arrangements will be apparent to those of skill in the art upon reviewing the above description. Other arrangements may be utilized and derived therefrom, such that structural and logical substitutions and changes may be made without departing from the spirit and scope of this disclosure. Figures are also merely representational or, as indicated, schematic, and thus may not be drawn to scale. Certain proportions thereof may be exaggerated, while others may be minimized. Accordingly, the specification and drawings are to be regarded in illustrative rather than a restrictive sense.

What is claimed is:

1. A portable, pediatric respiratory rate sensing device comprising:
an accelerometer adapted to detect respiration of a child;
a contact surface operatively connected to the accelerometer, the contact surface adapted to be placed in operative contact with the child's chest;
a gel material at least partially coating the contact surface, the gel material selected from the group consisting of silicone, polyvinyl chloride, and latex; and
a portable, rechargeable power source electrically connected to the accelerometer;
wherein the area and weight associated with the contact surface, and the characteristics of the gel material, are selected to substantially stabilize the accelerometer during use to reduce spurious signals.

2. The device of claim 1, wherein the contact surface has an area about 4 square inches to about 14 square inches, a weight of about 130 to about 200 grams, and the gel material has an area substantially corresponding to the contact surface, a thickness of about ⅛″ to ¼″, and a tackiness sufficient, under standard atmospheric conditions, to both adhere to the skin of the child being examined and be harmlessly removable therefrom.

3. The device of claim 1, further comprising a housing carrying the accelerometer, wherein the power source comprises a manually operable crank charger, the charger being selectively connectable to the housing.

4. The device of claim 1, wherein the device configured to sense respiratory rates in children from newborn infants to age five.

5. The device of claim 1, further comprising a control system for the device, the control system including a user interface and a processor suitably programmed to process input from the user interface and from the accelerometer.

6. The device of claim 5, wherein the control system is configured to operate the device in three modes consisting of infant, toddler, and child modes.

7. The device of claim 1, further comprising a transmitter operatively connected to the accelerometer to send signals corresponding to respiration detected by the accelerometer in a format selected from a group consisting of Bluetooth, Wi-Fi, NFC, radio, cellular, AM/FM, 802.5.14, and protocols of wireless diagnostic devices.

8. The device of claim 1, wherein the gel material comprises silicone configured to be peeled away from the contact surface and remain substantially intact, washable, and replaceable back on a corresponding contact surface for reuse.

9. The device of claim 1, further comprising:
a user interface module having user input areas to select respiratory programs and a screen for displaying corresponding readouts;
a first sensor module including the accelerometer, and a first power module including the rechargeable power source;
wherein the first power module and the first sensor module are user-detachable to enable attachment of a second power module and a second sensor module, said second modules being different from the first modules.

10. The device of claim 9, wherein the screen comprises a touch screen and at least some of the user input areas are available via the touch screen, the user interface module having a lower surface at least partially comprising the contact surface.

11. The device of claim 9, wherein the user input areas comprise selection buttons corresponding to respective modes of operation selected from the group consisting of infant, toddler, and child.

12. The device of claim 1, further comprising a temperature sensor, the temperature sensor operatively connected to the contact surface.

13. The device of claim 1, wherein the contact surface includes a pair of resiliently flexible straps for laying out on the child's chest and wrapping around a wrist.

14. A hand-held pediatric medical diagnostic device, comprising:
a sensor module adapted to detect one or more corresponding medical conditions of a child;
a contact surface operatively connected to the sensor module, the contact surface adapted to be placed in operative contact with a child;
a control module including a user interface having user input areas to initiate at least one diagnostic program associated with the sensor module, and a processer suitably programmed to process input from the user interface and from the sensor module;
a portable, rechargeable power source electrically connected to the control module;
wherein the sensor module includes at least one sensor selected from the group consisting of an accelerometer, a thermometer, a galvanic sensor, a heart rate sensor, an oximeter, a stethoscope, a flow meter, an ECG/EKG sensor, an otoscope, and a photographic camera.

15. The device of claim 14, further comprising:
a gel material at least partially coating the contact surface, the gel material selected from the group consisting of silicone, polyvinyl chloride, and latex; and
wherein the area and weight associated with the contact surface, and the characteristics of the gel material, are selected to substantially stabilize the sensor module during use.

16. The device of claim 15, wherein the contact surface has an area about 10 square inches to about 14 square inches, a weight of about 130 grams to about 200 grams, and the gel material has an area substantially corresponding to the contact surface, a thickness of about ⅛ inch, and a tackiness sufficient, under standard atmospheric conditions, to both adhere to the skin of the child being examined and be harmlessly removable therefrom.

17. The device of claim 14, further comprising a housing carrying the control module, and wherein the power source comprises a manually operable crank charger, the charger selectively connectable to the housing.

18. The device of claim 14, further comprising a transmitter operatively connected to the control system to send signals corresponding to the medical condition detected by the sensor module in a format selected from the group consisting of Bluetooth, Wi-Fi, NFC, radio, cellular, AM/FM, 802.5.14, and protocols of wireless diagnostic devices.

19. The device of claim 14, further comprising a plurality of the sensor modules and a multiplexed sensor connector electrically connected to the control system, the connector adapted to removably receive any selected one of the sensor modules.
